(19) United States
(12) Patent Application Publication
(10) Pub. No.: US 2009/0068713 A1
(43) Pub. Date: Mar. 12, 2009

(54) PROCESS FOR PRODUCING GAMMA-GLUTAMYLAMIDE COMPOUNDS

(75) Inventors: Koichiro Miyake, Hofu-shi (JP); Shin-ichi Hashimoto, Hofu-shi (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., Chiyoda-ku, Tokyo (JP)

(21) Appl. No.: 11/913,472

(22) PCT Filed: May 9, 2006
(86) PCT No.: PCT/JP2006/309342

§ 371 Date: Nov. 2, 2007

(30) Foreign Application Priority Data
May 9, 2005 (JP) 2005-136662

(51) Int. Cl. C12P 13/14

(52) U.S. Cl. 435/110

(57) ABSTRACT

The present invention provides a process for producing a γ-glutamylamide compound, which comprises forming the γ-glutamylamide compound from a glutamyl donor and an amine compound using γ-glutamylcysteine synthetase, preferably γ-glutamylcysteine synthetase derived from a microorganism, or a culture of cells having the enzyme or a treated culture as an enzyme source.

### TECHNICAL FIELD

The present invention relates to a process for producing a γ-glutamylamide compound using γ-glutamylcysteine synthetase, or a culture of cells having the enzyme or a treated culture as an enzyme source.

### BACKGROUND ART

γ-Glutamylcysteine synthetase is known as an enzyme synthesizing γ-glutamyl peptides (see non-patent document No. 1). It is known that γ-glutamylcysteine synthetase has relatively broad substrate specificity and has the activity to form various γ-glutamyl amino acids from L-glutamic acid and various amino acids (see patent document No. 1).

It is also known that γ-glutamylcysteine synthetase has the activity to form γ-glutamyl-4-hydroxyanilide from, other than amino acids, 4-hydroxyaniline and L-glutamic acid (see patent document No. 2).

However, it is not known that γ-glutamylcysteine synthetase has the activity to form a γ-glutamylamide compound from a glutamyl donor such as L-glutamic acid and an amine compound such as ethylamine.

Theanine, a kind of γ-glutamylamide compound, is known as the main component of umami of gyokuro tea, a premium variety of green tea, and is an important substance as a flavoring ingredient of tea and other foods.

Theanine is suggested to have various physiological effects including relaxation effect, improvement of sleep disturbance, suppression of blood pressure elevation, improvement of sensitivity to cold, prevention of epilepsy and better concentration, and is regarded as a promising material for health foods.

As the enzymatic methods for producing theanine, a method using glutaminase (see patent document No. 3), a method using γ-glutamyltranspeptidase (see patent document No. 4), a method using ipuC (γ-glutamylamide synthetase) derived from a bacterium belonging to the genus Pseudomonas (see patent document No. 5), etc. are known. Although the method using glutaminase is the only method that has been put to practical application, it involves problems such that it is necessary to carry out a reaction under highly alkaline conditions at high ethylamine concentration as it uses the reverse reaction of normal enzymatic reaction and that the yield is low because the substrate glutamine is converted to glutamic acid through hydrolyzation reaction by glutaminase. Thus, more efficient production methods are desired.

Non-patent document No. 1:
Seimei Kogaku Kenkyu Report, Kumagai et al., Vol. 14, No. 4, 8-17 (1985)
   Patent document No. 1:
Japanese Published Unexamined Patent Application No. 132896/82
   Patent document No. 2:
Japanese Published Unexamined Patent Application No. 99197/82
   Patent document No. 3:
Japanese Published Unexamined Patent Application No. 225789/99
   Patent document No. 4:
Japanese Published Unexamined Patent Application No. 89266/96
   Patent document No. 5:
WO01/73038 pamphlet

### DISCLOSURE OF THE INVENTION

### Problems to be Solved by the Invention

An object of the present invention is to provide a simple and efficient process for producing a glutamylamide compound, preferably theanine.

### Means for Solving the Problems

The present invention relates to the following (1) to (10).
(1) A process for producing a γ-glutamylamide compound represented by formula (I):

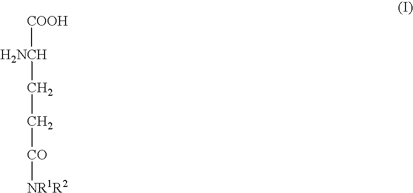

(wherein R¹ and R², which may be the same or different, each represent a hydrogen atom, substituted or unsubstituted lower alkyl, substituted or unsubstituted lower alkenyl, or substituted or unsubstituted lower alkynyl, but are not hydrogen atoms at the same time), which comprises forming the γ-glutamylamide compound from a glutamyl donor and an amine compound using γ-glutamylcysteine synthetase (E.C.6.3.2.2), or a culture of cells having the enzyme or a treated culture the cells as an enzyme source.
(2) The process according to the above (1), wherein the γ-glutamylcysteine synthetase is an enzyme derived from a microorganism.
(3) The process according to the above (2), wherein the microorganism is any of an enteric bacterium, yeast and a filamentous fungus.
(4) The process according to the above (2), wherein the microorganism is a microorganism belonging to the genus Escherichia.
(5) The process according to the above (1), wherein the γ-glutamylcysteine synthetase is a protein according to any of the following [1] to [3]:
   [1] a protein having the amino acid sequence of SEQ ID NO: 1;
   [2] a protein consisting of an amino acid sequence wherein one or more amino acid residues are deleted, substituted or added in the amino acid sequence of SEQ ID NO: 1 and having γ-glutamylcysteine synthetase activity; and
   [3] a protein consisting of an amino acid sequence which has 80% or more homology to the amino acid of SEQ ID NO: 1.
(6) The process according to the above (1), wherein the cell is a microorganism.
(7) The process according to the above (6), wherein the microorganism is any of an enteric bacterium, yeast and a filamentous fungus.
(8) The process according to the above (6), wherein the microorganism is a microorganism belonging to the genus Escherichia.
(9) The process according to any one of the above (1) and (6) to (8), wherein the cell having γ-glutamylcysteine synthetase is a cell into which a polynucleotide encoding γ-glutamylcysteine synthetase has been introduced.
(10) The process according to the above (9), wherein the polynucleotide encoding γ-glutamylcysteine synthetase is a polynucleotide according to any of the following [1] to [3]:
   [1] a polynucleotide encoding the protein according to any of [1] to [3] of the above (5);
   [2] a polynucleotide having the nucleotide sequence of SEQ ID NO: 2; and
   [3] a polynucleotide which hybridizes with a polynucleotide having a nucleotide sequence complementary to the nucleotide sequence of SEQ ID NO: 2 under stringent conditions and which encodes a protein having γ-glutamylcysteine synthetase activity.

### EFFECT OF THE INVENTION

In accordance with the present invention, γ-glutamylamide compounds, preferably theanine can be produced simply and efficiently.

### BRIEF DESCRIPTION OF THE DRAWINGS

FIG. 1 shows the structure of plasmid pGSK1 that expresses the γ-glutamylcysteine synthetase gene derived from Escherichia coli W3110.

### EXPLANATION OF SYMBOLS

gsh I: γ-glutamylcysteine synthetase gene derived from Escherichia coli W3110
Plac: lactose operon promoter region
Tlpp: terminator sequence of lipoprotein derived from Escherichia coli
Ap: ampicillin resistance gene

### BEST MODES FOR CARRYING OUT THE INVENTION

### 1. γ-Glutamylamide Compounds Produced by the Process of the Present Invention

γ-Glutamylamide compounds produced by the process of the present invention include those represented by formula (I) (wherein R¹ and R², which may be the same or different, each represent a hydrogen atom, substituted or unsubstituted lower alkyl, substituted or unsubstituted lower alkenyl, or substituted or unsubstituted lower alkynyl, but are not hydrogen atoms at the same time).

In the definition of formula (I), the lower alkyl includes straight- or branched-chain alkyl or cyclic alkyl or alkyl comprising a combination thereof having 1 to 10 carbon atoms. Specific examples of the straight- and branched-chain alkyl are methyl, ethyl, n-propyl, isopropyl, n-butyl, isobutyl, sec-butyl, tert-butyl, n-pentyl, neopentyl, n-hexyl, n-heptyl, n-octyl, n-nonyl and n-decyl. Examples of the cyclic alkyl are cyclopropyl, cyclobutyl, cyclopentyl, cyclohexyl, cycloheptyl, cyclooctyl, cyclodecyl, noradamantyl and adamantyl. Examples of the alkyl comprising a combination of straight- or branched-chain alkyl and cyclic alkyl are cyclopropylmethyl, cyclopentylmethyl and cyclooctylethyl.

The lower alkenyl includes straight- or branched-chain alkenyl having 2 to 10 carbon atoms such as vinyl, allyl, 1-propenyl, 1-butenyl, 3-butenyl, 2-pentenyl, 4-pentenyl, 2-hexenyl, 5-hexenyl, 1-heptenyl, 4-heptenyl, 6-heptenyl, 2-decenyl, 1-octenyl, 9-decenyl, 1-nonenyl and 6-nonenyl.

The lower alkynyl includes straight- or branched-chain alkenyl having 2 to 10 carbon atoms such as ethynyl, propynyl, butynyl, pentynyl, hexynyl, heptynyl, octynyl, nonynyl and decynyl.

The substituted lower alkyl, the substituted lower alkenyl and the substituted lower alkynyl have 1 to a substitutable number of substituents, preferably 1 to 3 substituents, more preferably 1 substituent, which are the same or different, such as halogen, nitro and hydroxy.

Preferred γ-glutamylamide compounds produced by the process of the present invention include those in which R¹ of formula (I) is a hydrogen atom and R² is methyl, ethyl, propyl, cyclopropyl or butyl, and more preferred is theanine represented by formula (II) below:

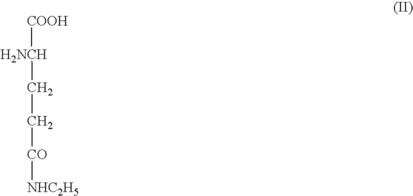

### 2. γ-Glutamylcysteine Synthetase Used in the Present Invention

The γ-glutamylcysteine synthetase used in the present invention may be γ-glutamylcysteine synthetase of any origin, and preferably includes the enzyme derived from enteric bacteria, yeast or filamentous fungi. Specific examples are the protein derived from Arabidopsis thaliana and having the amino acid sequence registered under the swiss-prot accession number P46309 (hereinafter, the number preceding the name of organisms likewise designates a swiss-prot accession number and means that it is a protein having the amino acid sequence registered under the accession number), Q7WES2 derived from Bordetella bronchiseptica, Q7W3F2 derived from Bordetella parapertussis, O23736 derived from Brassica juncea, P57485, P58994 and Q89AD8 derived from Buchnera aphidicola, Q20117 derived from Caenorhabditis elegans, Q9HF78 derived from Candida albicans, Q971V1 derived from Clostridium acetobutylicum, derived from Clostridium perfringens, Q9W3K5 derived from Drosophila melanogaster, Q8×900, P06980, 1501198A and CAA27583 derived from Escherichia coli, Q82ZG8 derived from Enterococcus faecalis, Q8F4D5 derived from Leptospira interrogans, Q926X7 derived from Listeria innocua, Q8Y3R3 derived from Listeria monocytogenes, O22493 derived from Lycopersicon esculentum, Q9ZNX6 derived from Medicago truncatula, Q8X0X0 derived from Neurospora crassa, Q9NFN6 derived from Onchocerca volvulus, Q9CM00 derived from Pasteurella multocida, Q7N7A4 derived from Photorhabdus luminescens, Q9HTY6 derived from Pseudomonas aeruginosa<, Q88R90 derived from Pseudomonas putida, Q88AR1 and P61379 derived from Pseudomonas syringae, Q8Z4D6 derived from Salmonella typhi, O68838 derived from Salmonella typhimurium, Q09768 derived from Schizosaccharomyces pombe, Q8EBF9 derived from Shewanella oneidensis, Q8E399 derived from Streptococcus agalactiae, Q8DXM9 derived from Streptococcus agalactiae, Q8DW15 derived from Streptococcus mutans, Q9KUG5 derived from Vibrio cholerae, Q87LS2 derived from Vibrio parahaemolyticus, Q8DC38 derived from Vibrio vulnificus, Q7 MHS5 derived from Vibrio vulnificus, Q8D2V5 derived from Wigglesworthia glossinidia, P32477 derived from Saccharomyces cerevisiae, Q8ZBU2 derived from Yersinia pestis, P48507 and P48506 derived from Homo sapiens, P97494 derived from Mus musculus, and P48508 and P19468 derived from Rattus norvegicus.

Suitable γ-glutamylcysteine synthetase used in the present invention is more preferably the enzyme derived from microorganisms among the above enzyme, further preferably the enzyme derived from enteric bacteria, yeast or filamentous fungi, particularly preferably the enzyme derived from Escherichia coli, and most preferably a protein having the amino acid sequence of SEQ ID NO: 1.

The γ-glutamylcysteine synthetase used in the present invention also includes a protein consisting of an amino acid sequence wherein one or more amino acid residues are deleted, substituted or added in the amino acid sequence of SEQ ID NO: 1 and having γ-glutamylcysteine synthetase activity.

The above protein consisting of an amino acid sequence wherein one or more amino acid residues are deleted, substituted or added and having γ-glutamylcysteine synthetase activity can be obtained, for example, by introducing a site-directed mutation into DNA encoding a protein consisting of the amino acid sequence of SEQ ID NO: 1 by site-directed mutagenesis described in Molecular Cloning, A Laboratory Manual, Third Edition, Cold Spring Harbor Laboratory Press (1989) (hereinafter referred to as Molecular Cloning, Third Edition); Current Protocols in Molecular Biology, John Wiley & Sons (1987-1997) (hereinafter referred to as Current Protocols in Molecular Biology); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc.

The number of amino acid residues which are deleted, substituted or added is not specifically limited, but is within the range where deletion, substitution or addition is possible by known methods such as the above site-directed mutagenesis. The suitable number is 1 to dozens, preferably 1 to 20, more preferably 1 to 10, further preferably 1 to 5.

The expression “one or more amino acid residues are deleted, substituted or added in the amino acid sequence of SEQ ID NO: 1” means that the amino acid sequence may contain deletion, substitution or addition of a single or plural amino acid residues at an arbitrary position therein.

Amino acid residues that may be substituted are, for example, amino acids which are not conserved in all of the amino acid sequences when the amino acid sequence of SEQ ID NO: 1 is compared with those of the γ-glutamylcysteine synthetase derived from the above-described various organisms using known alignment-software. An example of known alignment software is alignment analysis software contained in gene analysis software Genetyx (Software Development Co., Ltd.). As analysis parameters for the analysis software, default values can be used.

Deletion or addition of amino acid residues may be contained, for example, in the N-terminal or C-terminal region of the amino acid sequence of SEQ ID NO: 1.

Deletion, substitution and addition may be simultaneously contained in one sequence, and amino acids to be substituted or added may be either natural or not. Examples of the natural amino acids are L-alanine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine.

The following are examples of the amino acids capable of mutual substitution. The amino acids in the same group can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

The protein used in the present invention includes a protein consisting of an amino acid sequence which has 80% or more homology, preferably 90% or more homology, more preferably 95% or more homology, further preferably 97% or more homology, particularly preferably 98% or more homology and most preferably 99% or more homology to the amino acid sequence of SEQ ID NO: 1 and having γ-glutamylcysteine synthetase activity.

The homology among amino acid sequences and nucleotide sequences can be determined by using algorithm BLAST by Karlin and Altschul [Pro. Natl. Acad. Sci. USA, 90, 5873 (1993)] and FASTA [Methods Enzymol., 183, 63 (1990)]. On the basis of the algorithm BLAST, programs such as BLASTN and BLASTX have been developed [J. Mol. Biol., 215, 403 (1990)]. When a nucleotide sequence is analyzed by BLASTN on the basis of BLAST, the parameters, for instance, are as follows: score=100 and wordlength=12. When an amino acid sequence is analyzed by BLASTX on the basis of BLAST, the parameters, for instance, are as follows: score=50 and wordlength=3. When BLAST and Gapped BLAST programs are used, default parameters of each program are used. The specific techniques for these analyses are known (http://www.ncbi.nlm.nih.gov.).

It is possible to confirm that the protein consisting of an amino acid sequence wherein one or more amino acid residues are deleted, substituted or added in the amino acid sequence of SEQ ID NO: 1 is a protein having γ-glutamylcysteine synthetase activity, for example, in the following manner. That is, a transformant expressing the protein whose enzymatic activity is to be confirmed is prepared by recombinant DNA techniques, the protein is produced using the transformant, and then the protein, L-glutamic acid and L-cysteine are allowed to be present in an aqueous medium, followed by HPLC analysis or the like to know whether γ-glutamylcysteine is formed and accumulated in the aqueous medium.

### 3. Cells Used in the Present Invention

The cells used in the present invention may be either microorganism cells, or animal or plant cells so long as they have γ-glutamylcysteine synthetase, and preferably include the above cells having a polynucleotide encoding γ-glutamylcysteine synthetase.

Examples of the cells are those of the various organisms having γ-glutamylcysteine synthetase of the above 2, preferably microorganisms among the cells, more preferably enteric bacteria, yeast and filamentous fungi among the microorganisms, and further preferably Escherichia coli.

Further, the cells used in the present invention are preferably those in which γ-glutamylcysteine synthetase activity is enhanced.

The cells in which γ-glutamylcysteine synthetase activity is enhanced include mutant strains obtained by treating the cell having γ-glutamylcysteine synthetase of the above 2 with a mutagen, for example, N-methyl-N′-nitro-N-nitrosoguanidine (NTG) by known methods and selecting the strains in which γ-glutamylcysteine synthetase activity is enhanced compared with the cell before mutation, and recombinant strains obtained by introducing a polynucleotide encoding γ-glutamylcysteine synthetase into the cell using recombinant techniques.

Suitable cells used in the present invention are preferably those having γ-glutamylcysteine synthetase and having the ability to produce a glutamyl donor, for example, L-glutamic acid, more preferably those having γ-glutamylcysteine synthetase, in which the ability to produce a glutamyl donor, for example, L-glutamic acid is enhanced. Examples of such cells are microorganisms, preferably procaryotes, more preferably Escherichia coli, in which the ability to produce L-glutamic acid is artificially enhanced using known methods.

### 4. Polynucleotide Encoding γ-Glutamylcysteine Synthetase Used in the Present Invention

The polynucleotide used in the present invention is DNA or RNA, preferably DNA and may either be double- or single-stranded. If the polynucleotide is double-stranded, it may be double-strand DNA, double-strand RNA or DNA-RNA hybrid. If the polynucleotide is single-stranded, it may either be a sense strand (i.e., coding strand) or an antisense strand (i.e., non-coding strand).

The polynucleotide encoding γ-glutamylcysteine synthetase used in the present invention may be of any origin so long as it encodes γ-glutamylcysteine synthetase, and preferably includes those encoding γ-glutamylcysteine synthetase derived from the cells having γ-glutamylcysteine synthetase of the above 2. Specific examples are the polynucleotide derived from Arabidopsis thaliana and having the nucleotide sequence registered with GenBank under the accession number Z29490 (hereinafter, the number preceding the name of organisms designates a GenBank accession number and means that it is a polynucleotide having the nucleotide sequence registered under the accession number), BX640450 derived from Bordetella bronchiseptica, BX640435 derived from Bordetella parapertussis, Y10848 derived from Brassica juncea, BA000003, AE014115 and AE014017 derived from Buchnera aphidicola, Z54218 derived from Caenorhabditis elegans, AF176677 derived from Candida albicans, AE007664 derived from Clostridium acetobutylicum, BA000016 derived from Clostridium perfringens, AF244351 derived from Drosophila melanogaster, AE005497, AE016765 and X03954 derived from Escherichia coli, AE016956 derived from Enterococcus faecalis, AE011382 derived from Leptospira interrogans, AL596174 derived from Listeria innocua, AL591984 derived from Listeria monocytogenes, AF017983 derived from Lycopersicon esculentum, AF041340 derived from Medicago truncatula, AL670009 derived from Neurospora crassa, AF042168 derived from Onchocerca volvulus, AE006146 derived from Pasteurella multocida, BX571863 derived from Photorhabdus luminescens, AE004933 derived from Pseudomonas aeruginosa, AE016774 derived from Pseudomonas putida, AE016857 and AY374326 derived from Pseudomonas syringae, AL627276 derived from Salmonella typhi, AF055352 derived from Salmonella typhimurium, X85017 derived from Schizosaccharomyces pombe, AE015792 derived from Shewanella oneidensis, AL766854 derived from Streptococcus agalactiae, AE014274 derived from Streptococcus agalactiae, AE014876 derived from Streptococcus mutans, AE004141 derived from Vibrio cholerae, BA000031 derived from Vibrio parahaemolyticus, AE016802 derived from Vibrio vulnificus, BA000037 derived from Vibrio vulnificus, BA000021 derived from Wigglesworthia glossinidia, D90220 derived from Saccharomyces cerevisiae, AJ414156 derived from Yersinia pestis, L35546 and 1490856 derived from Homo sapiens, U85414 derived from Mus musculus, and S65555 and J05181 derived from Rattus norvegicus.

Suitable polynucleotides encoding γ-glutamylcysteine synthetase used in the present invention are more preferably those derived from the above microorganisms, further more preferably those derived from enteric bacteria, yeast or filamentous fungi among said microorganisms, particularly preferably those derived from microorganisms belonging to the genus Escherichia among said enteric bacteria, particularly preferably those derived from Escherichia coli, and most preferably a polynucleotide having the nucleotide sequence of SEQ ID NO: 2.

The polynucleotide encoding γ-glutamylcysteine synthetase used in the present invention also includes polynucleotides which hybridize with a polynucleotide having a nucleotide sequence complementary to the nucleotide sequence of SEQ ID NO: 2 under stringent conditions and which encode a protein having γ-glutamylcysteine synthetase activity.

“To hybridize” refers to hybridization of a polynucleotide with a polynucleotide having a specific nucleotide sequence or a part thereof. Therefore, the polynucleotide having a specific nucleotide sequence or a part thereof is a polynucleotide which can be used as a probe for Northern or Southern blot analysis or as an oligonucleotide primer for PCR analysis. Polynucleotides used as a probe include polynucleotides consisting of at least 100 nucleotides, preferably 200 or more nucleotides, more preferably 500 or more nucleotides, and those used as a primer include polynucleotides consisting of at least 10 nucleotides, preferably 15 or more nucleotides.

The method for hybridization of a polynucleotide is well known, and persons skilled in the art, for example, can determine the conditions for hybridization according to the present specification. The conditions for the hybridization can be determined and the hybridization can be carried out according to the methods described in Molecular Cloning, Second Edition, Third Edition (2001); Methods for General and Molecular Bacteriology, ASM Press (1994); Immunology methods manual, Academic press (Molecular), and many other standard textbooks.

Hybridization under the above stringent conditions is carried out, preferably, as follows. A filter with a polynucleotide, preferably DNA immobilized thereon and a probe, preferably probe DNA are incubated in a solution comprising 50% formamide, 5×SSC (750 mM sodium chloride and 75 mM sodium citrate), 50 mM sodium phosphate (pH 7.6), 5×Denhardt's solution, 10% dextran sulfate and 20 μg/l denatured salmon sperm DNA at 42° C. overnight, and after the incubation, the filter is washed in 0.2×SSC solution (ca. 65° C.). Less stringent conditions can also be employed. Modification of the stringent conditions can be made by adjusting the concentration of formamide (the conditions become less stringent as the concentration of formamide is lowered) and by changing the salt concentrations and the temperature conditions. Hybridization under less stringent conditions is carried out, for example, by incubating the above filter and probe in a solution comprising 6×SSCE (20×SSCE: 3 mol/l sodium chloride, 0.2 mol/l sodium dihydrogenphosphate and 0.02 mol/l EDTA, pH 7.4), 0.5% SDS, 30% formamide and 100 μg/l denatured salmon sperm DNA at 37° C. overnight, and washing the filter with 1×SSC solution containing 0.1% SDS (50° C.). Hybridization under still less stringent conditions is carried out by using a solution having a high salt concentration (for example, 5×SSC) under the above less stringent conditions, followed by washing.

Various conditions described above can also be established by adding a blocking reagent used to reduce the background of hybridization or changing the reagent. The addition of the above blocking reagent may be accompanied by changes of conditions for hybridization to make the conditions suitable for the purpose.

The polynucleotide capable of hybridization under stringent conditions described above includes polynucleotides having at least 90% homology, preferably 95% or more homology, more preferably 97% or more homology, further preferably 98% or more homology, particularly preferably 99% or more homology to the nucleotide sequence of SEQ ID NO: 2 as calculated by use of BLAST and FASTA described above based on the above parameters.

It is possible to confirm that the polynucleotide hybridizing with a polynucleotide having the nucleotide sequence of SEQ ID NO: 2 under stringent conditions is a polynucleotide encoding a protein having γ-glutamylcysteine synthetase activity, for example, by preparing a protein encoded by the polynucleotide using recombinant techniques and measuring the activity of the protein as mentioned above.

### 5. Process for Preparing γ-Glutamylcysteine Synthetase Used in the Present Invention

γ-Glutamylcysteine synthetase used in the present invention can be obtained by culturing the cell of the above 3 in a medium, allowing γ-glutamylcysteine synthetase to form and accumulate in the culture, and separating and purifying the γ-glutamylcysteine synthetase from the culture according to known methods for purifying proteins. Preferably, the enzyme can be obtained by a method using, as the cell having γ-glutamylcysteine synthetase, a recombinant cell in which γ-glutamylcysteine synthetase activity is enhanced, which is obtained by introducing a polynucleotide encoding γ-glutamylcysteine synthetase into a cell using recombinant techniques, and obtaining the enzyme from the culture of the recombinant cell.

### (1) Process for Preparing a Polynucleotide Encoding γ-Glutamylcysteine Synthetase

The polynucleotide encoding γ-glutamylcysteine synthetase used in the present invention can be obtained, for example, by Southern hybridization of the chromosomal DNA library from each organism using a probe DNA which can be designed based on the nucleotide sequence of the polynucleotide encoding γ-glutamylcysteine synthetase of the above 4, or by PCR [PCR Protocols, Academic Press (1990)] using primer DNAs which can be designed based on said nucleotide sequence, and as a template, the chromosomal DNA of the organisms of the above 2, etc.

The polynucleotide encoding γ-glutamylcysteine synthetase can also be obtained by conducting a search through various gene sequence databases for a sequence having 85% or more homology, preferably 90% or more homology, more preferably 95% or more homology, further preferably 97% or more homology, particularly preferably 98% or more homology, most preferably 99% or more homology to the nucleotide sequence of the polynucleotide encoding γ-glutamylcysteine synthetase of the above 4, and obtaining the polynucleotide encoding γ-glutamylcysteine synthetase, based on the nucleotide sequence obtained by the search, from a chromosomal DNA or cDNA library of an organism having the nucleotide sequence according to the above methods.

The obtained polynucleotide, as such or after cleavage with appropriate restriction enzymes, is inserted into a vector by a conventional method, and the obtained recombinant DNA is introduced into a host cell. Then, the nucleotide sequence of the polynucleotide can be determined by a conventional sequencing method such as the dideoxy method [Proc. Natl. Acad. Sci., USA, 74, 5463 (1977)] or by using a nucleotide sequencer such as 373A DNA Sequencer (Perkin-Elmer Corp.).

In cases where the obtained polynucleotide is found to be a partial polynucleotide by the analysis of nucleotide sequence, the full length polynucleotide can be obtained by Southern hybridization of a chromosomal DNA library using the partial polynucleotide as a probe.

It is also possible to prepare the desired polynucleotide by chemical synthesis using a DNA synthesizer (e.g., Model 8905, PerSeptive Biosystems) based on the determined nucleotide sequence of the polynucleotide.

An example of the polynucleotide that can be obtained by the above-described method is a polynucleotide having the nucleotide sequence of SEQ ID NO: 2.

Examples of the vectors for inserting the polynucleotide encoding γ-glutamylcysteine synthetase include pBluescript II KS(+) (Stratagene), pDIRECT [Nucleic Acids Res., 18, 6069 (1990)], pCR-Script Amp SK(+) (Stratagene), pT7Blue (Novagen, Inc.), pCR II (Invitrogen Corp.) and pCR-TRAP (GenHunter Corp.).

As the host cell, microorganisms belonging to the genus Escherichia, etc. can be used. Examples of the microorganisms belonging to the genus Escherichia include Escherichia coli XL1-Blue, Escherichia coli XL2-Blue, Escherichia coli DH1, Escherichia coli MC1000, Escherichia coli ATCC 12435, Escherichia coli W1485, Escherichia coli JM109, Escherichia coli HB101, Escherichia coli No. 49, Escherichia coli W3110, Escherichia coli NY49, Escherichia coli MP347, Escherichia coli NM522, Escherichia coli BL21 and Escherichia coli ME8415.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

An example of the transformant obtained by the above method is Escherichia coli BL21/pGSK1, which is a microorganism carrying a recombinant DNA comprising a polynucleotide having the nucleotide sequence of SEQ ID NO: 2.

### (2) Process for Preparing the Transformant Having γ-Glutamylcysteine Synthetase

On the basis of the polynucleotide encoding γ-glutamylcysteine synthetase obtained by the process of the above (1), a DNA fragment of an appropriate length comprising a region encoding γ-glutamylcysteine synthetase is prepared according to need. A transformant having enhanced productivity of the enzyme can be obtained by replacing a nucleotide in the nucleotide sequence of the region encoding γ-glutamylcysteine synthetase so as to make a codon most suitable for the expression in a host cell.

The DNA fragment is inserted downstream of a promoter in an appropriate expression vector to prepare a recombinant DNA.

A transformant which produces γ-glutamylcysteine synthetase can be obtained by introducing the recombinant DNA into a host cell suited for the expression vector.

As the host cell, any bacterial cells, yeast cells, animal cells, insect cells, plant cells, etc. that are capable of expressing the desired polynucleotide can be used.

The expression vectors that can be employed are those capable of autonomous replication or integration into the chromosome in the above host cells and comprising a promoter at a position appropriate for the transcription of the DNA encoding γ-glutamylcysteine synthetase.

When a procaryote such as a bacterium is used as the host cell, it is preferred that the recombinant DNA comprising the DNA encoding γ-glutamylcysteine synthetase is a recombinant DNA which is capable of autonomous replication in the procaryote and which comprises a promoter, a ribosome binding sequence, the DNA encoding γ-glutamylcysteine synthetase and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelix1 (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 (Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Shuzo Co., Ltd.), pUC118 (Takara Shuzo Co., Ltd.) and pPA1 (Japanese Published Unexamined Patent Application No. 233798/88).

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, SPO1 promoter, SPO2 promoter and penP promoter can be used. Artificially designed and modified promoters such as a promoter in which two Pₜᵣₚs are combined in tandem, tac promoter, lacT7 promoter and letI promoter, etc. can also be used.

Also useful are promoters such as xylA promoter for the expression in microorganisms belonging to the genus Bacillus [Appl. Microbiol. Biotechnol., 35, 594-599 (1991)] and P54-6 promoter for the expression in microorganisms belonging to the genus Corynebacterium [Appl. Microbiol. Biotechnol., 53, 674-679 (2000)].

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA encoding γ-glutamylcysteine synthetase is ligated to an expression vector, the transcription termination sequence is not essential, but it is preferred to place the transcription termination sequence immediately downstream of the structural gene.

An example of such recombinant DNA is pGSK1.

Examples of procaryotes include microorganisms belonging to the genera Escherichia, Serratia, Bacillus, Brevibacterium, Corynebacterium, Microbacterium, Pseudomonas, Agrobacterium, Alicyclobacillus, Anabaena, Anacystis, Arthrobacter, Azotobacter, Chromatium, Erwinia, Methylobacterium, Phormidium, Rhodobacter, Rhodopseudomonas, Rhodospirillum, Scenedesmus, Streptomyces, Synechoccus and Zymomonas. Specific examples are Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia Coli MP347, Escherichia coli NM522, Escherichia coli BL21, Bacillus subtilis ATCC 33712, Bacillus megaterium, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 14297, Corynebacterium acetoacidophilum ATCC 13870, Microbacterium ammoniaphilum ATCC 15354, Serratia ficaria, Serratia fonticola, Serratia liquefaciens; Serratia marcescens, Pseudomonas sp. D-0110, Agrobacterium radiobacter, Agrobacterium rhizogenes, Agrobacterium rubi, Anabaena cylindrica, Anabaena doliolum, Anabaena flos-aquae, Arthrobacter aurescens, Arthrobacter citreus, Arthrobacter globiformis, Arthrobacter hydrocarboglutamicus, Arthrobacter mysorens, Arthrobacter nicotianae, Arthrobacter paraffineus, Arthrobacter protophormiae, Arthrobacter roseoparaffinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Erwinia herbicola, Erwinia punctata, Erwinia terreus, Methylobacterium rhodesianum, Methylobacterium extorquens, Phormidium sp. ATCC 29409, Rhodobacter capsulatus, Rhodobacter sphaeroides, Rhodopseudomonas blastica, Rhodopseudomonas marina, Rhodopseudomonas palustris, Rhodospirillum rubrum, Rhodospirillum salexigens, Rhodospirillum salinarum, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus and Zymomonas mobilis.

Examples of suitable procaryotes are preferably microorganisms belonging to the genus Escherichia or Corynebacterium, more preferably Escherichia coli and Corynebacterium glutamicum.

Further, examples of suitable microorganisms are preferably those in which productivity of a glutamyl donor is enhanced, more preferably those in which productivity of L-glutamic acid is enhanced.

Specific examples are microorganisms, preferably procaryotes, more preferably microorganisms belonging to the genus Escherichia or Corynebacterium, further preferably Escherichia coli and Corynebacterium glutamicum to which the ability to produce L-glutamic acid has been artificially given by a known method.

Examples of the known methods include:
(a) a method in which at least one of the mechanisms regulating the biosynthesis of L-glutamic acid is partially released or completely released;
(b) a method in which the expression of at least one of the enzymes involved in the biosynthesis of L-glutamic acid is enhanced;
(c) a method in which the copy number of at least one of the enzyme genes involved in the biosynthesis of L-glutamic acid is increased;
(d) a method in which at least one of the metabolic pathways branching from the biosynthetic pathway of L-glutamic acid into metabolites other than L-glutamic acid is weakened or blocked; and
(e) a method in which a cell strain having a higher resistance to an analogue of L-glutamic acid as compared with a wild-type strain is selected.

The above known methods can be used alone or in combination.

Specific methods of the above (a) are described in Agric. Biol. Chem., 43, 105-111 (1979), J. Bacteriol., 110, 761-763 (1972), Appl. Microbiol. Biotechnol., 39, 318-323 (1993), etc. Specific methods of the above (b) are described in Agric. Biol. Chem., 43, 105-111 (1979), J. Bacteriol., 110, 761-763 (1972), etc. Specific methods of the above (c) are described in Appl. Microbiol. Biotechnol., 39, 318-323 (1993), Agric. Biol. Chem., 39, 371-377 (1987), etc. Specific methods of the above (d) are described in Appl. Environ. Microbiol., 38, 181-190 (1979), Agric. Biol. Chem., 42, 1773-1778 (1978), etc. Specific methods of the above (e) are described in Agric. Biol. Chem., 36, 1675-1684 (1972), Agric. Biol. Chem., 41, 109-116 (1977), Agric. Biol. Chem., 37, 2013-2023 (1973), Agric. Biol. Chem., 51, 2089-2094 (1987), etc. Microorganisms having the ability to produce L-glutamic acid can be prepared by referring to the above publications.

Further, processes for the preparation of microorganisms having the ability to produce L-glutamic acid by the methods of the above (a) to (e), alone or in combination, are described in Biotechnology 2nd ed., Vol. 6, Products of Primary Metabolism (VCH Verlagsgesellschaft mbH, Weinheim, 1996) section 14a, 14b; Advances in Biochemical Engineering/Biotechnology 79, 1-35 (2003); Hiroshi Soda, et al., Amino Acid Fermentation, Gakkai Shuppan Center (1986), etc., and microorganisms having the ability to produce L-glutamic acid can be prepared by referring to the above publications.

Many microorganisms having the ability to produce L-glutamic acid, for example, FERM BP-5807 and ATCC 13032 have been reported.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When a yeast strain is used as the host cell, YEp13 (ATCC 37115), YEp24 (ATCC 37051), YCp50 (ATCC 37419), pHS19, pHS15, etc. can be used as the expression vector.

As the promoter, any promoters capable of functioning in yeast strains can be used. Suitable promoters include PHO5 promoter, PGK promoter, GAP promoter, ADH promoter, gal 1 promoter, gal 10 promoter, heat shock polypeptide promoter, MFα1 promoter and CUP 1 promoter.

Examples of suitable host cells are yeast strains belonging to the genera Saccharomyces, Schizosaccharomyces, Kluyveromyces, Trichosporon, Schwanniomyces, Pichia and Candida, specifically, Saccharomyces cerevisiae, Schizosaccharomyces pombe, Kluyveromyces lactis, Trichosporon pullulans, Schwanniomyces alluvius, Pichia pastoris and Candida utilis.

Introduction of the recombinant DNA can be carried out by any of the methods for introducing DNA into yeast, for example, electroporation [Methods Enzymol., 194, 182 (1990)], the spheroplast method [Proc. Natl. Acad. Sci. USA, 81, 4889 (1984)] and the lithium acetate method [J. Bacteriol., 153, 163 (1983)].

When an animal cell is used as the host cell, pcDNAI, pcDM8 (commercially available from Funakoshi Co., Ltd.), pAGE107 (Japanese Published Unexamined Patent Application No. 22979/91), pAS3-3 (Japanese Published Unexamined Patent Application No. 227075/90), pCDM8 [Nature, 329, 840 (1987)], pcDNAI/Amp (Invitrogen Corp.), pREP4 (Invitrogen Corp.), pAGE103 [J. Biochem., 101, 1307 (1987)], pAGE210, pAMo, pAMoA, etc. can be used as the expression vector.

As the promoter, any promoters capable of functioning in animal cells can be used. Suitable promoters include the promoter of IE (immediate early) gene of cytomegalovirus (CMV), SV40 early promoter, metallothionein promoter, the promoter of a retrovirus, heat shock promoter, SRα promoter, etc. The enhancer of IE gene of human CMV may be used in combination with the promoter.

Examples of suitable host cells are mouse myeloma cells, rat myeloma cells, mouse hybridomas, human-derived Namalwa cells and Namalwa KJM-1 cells, human embryonic kidney cells, human leukemia cells, African green monkey kidney cells, Chinese hamster-derived CHO cells, and HBT5637 (Japanese Published Unexamined Patent Application No. 299/88).

The mouse myeloma cells include SP2/0 and NSO; the rat myeloma cells include YB2/0; the human embryonic kidney cells include HEK293 (ATCC CRL-1573); the human leukemia cells include BALL-1; and the African green monkey kidney cells include COS-1 and COS-7.

Introduction of the recombinant DNA can be carried out by any of the methods for introducing DNA into animal cells, for example, electroporation [Cytotechnology, 3, 133 (1990)], the calcium phosphate method (Japanese Published Unexamined Patent Application No. 227075/90), lipofection [Proc. Natl. Acad. Sci. USA, 84, 7413 (1987)], and the method described in Virology, 52, 456 (1973).

When an insect cell is used as the host cell, the protein can be produced by using the methods described in Baculovirus Expression Vectors, A Laboratory Manual, W. H. Freeman and Company, New York (1992); Current Protocols in Molecular Biology; Molecular Biology, A Laboratory Manual; Bio/Technology, 6, 47 (1988), etc.

That is, the recombinant gene transfer vector and a baculovirus are cotransfected into insect cells to obtain a recombinant virus in the culture supernatant of the insect cells, and then insect cells are infected with the recombinant virus, whereby the protein can be produced.

The gene transfer vectors useful in this method include pVL1392, pVL1393 and pBlueBacIII (products of Invitrogen Corp.).

An example of the baculovirus is Autographa californica nuclear polyhedrosis virus, which is a virus infecting insects belonging to the family Barathra.

Examples of the insect cells are ovarian cells of Spodoptera frugiperda, ovarian cells of Trichoplusia ni, and cultured cells derived from silkworm ovary.

The ovarian cells of Spodoptera frugiperda include Sf9 and Sf21 (Baculovirus Expression Vectors, A Laboratory Manual); the ovarian cells of Trichoplusia ni include High 5 and BTI-TN-5B1-4 (Invitrogen Corp.); and the cultured cells derived from silkworm ovary include Bombyx mori N4.

Cotransfection of the above recombinant gene transfer vector and the above baculovirus into insect cells for the preparation of the recombinant virus can be carried out by the calcium phosphate method (Japanese Published Unexamined Patent Application No. 227075/90), lipofection [Proc. Natl. Acad. Sci. USA, 84, 7413 (1987)], etc.

When a plant cell is used as the host cell, Ti plasmid, tobacco mosaic virus vector, etc. can be used as the expression vector.

As the promoter, any promoters capable of functioning in plant cells can be used. Suitable promoters include 35S promoter of cauliflower mosaic virus (CaMV), rice actin 1 promoter, etc.

Examples of suitable host cells are cells of plants such as tobacco, potato, tomato, carrot, soybean, rape, alfalfa, rice, wheat and barley.

Introduction of the recombinant vector can be carried out by any of the methods for introducing DNA into plant cells, for example, the method using Agrobacterium (Japanese Published Unexamined Patent Application Nos. 140885/84 and 70080/85, WO94/00977), electroporation (Japanese Published Unexamined Patent Application No. 251887/85) and the method using particle gun (gene gun) (Japanese Patent Nos. 2606856 and 2517813).

### (3) Process for Preparing γ-Glutamylcysteine Synthetase

γ-Glutamylcysteine synthetase can be produced by culturing the cells of the above 3 or the transformant obtained in the above (1) and (2) in a medium, allowing γ-glutamylcysteine synthetase to form and accumulate in the culture, and recovering the protein from the culture.

Culturing of the above cells and transformant having γ-glutamylcysteine synthetase in a medium can be carried out by conventional methods for culturing cells.

For the culturing of the cells of a procaryote such as Escherichia coli or a eucaryote such as yeast and the transformant obtained by using them as the host, any of natural media and synthetic media can be used insofar as it is a medium suitable for efficient culturing of the cells which contains carbon sources, nitrogen sources, inorganic salts, etc. which can be assimilated by the cells.

As the carbon sources, any carbon sources that can be assimilated by the organism can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

As the nitrogen sources, ammonia, ammonium salts of organic or inorganic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 40° C., and the culturing period is usually 5 hours to 7 days. The pH is maintained at 3.0 to 9.0 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with an expression vector comprising an inducible promoter is cultured, an inducer may be added to the medium, if necessary. For example, in the case of a microorganism transformed with an expression vector comprising lac promoter, isopropyl-β-D-thiogalactopyranoside or the like may be added to the medium; and in the case of a microorganism transformed with an expression vector comprising trp promoter, indoleacrylic acid or the like may be added.

For the culturing of animal cells and the transformant obtained by using an animal cell as the host cell, generally employed media such as RPMI1640 medium [J. Am. Med. Assoc., 199, 519 (1967)], Eagle's MEM [Science, 122, 501 (1952)], DMEM [Virology, 8, 396 (1959)] and 199 medium [Proc. Soc. Biol. Med., 73, 1 (1950)], media prepared by adding fetal calf serum or the like to these media, etc. can be used as the medium.

Culturing is usually carried out at pH 6 to 8 at 25 to 40° C. for 1 to 7 days in the presence of 5% CO₂.

If necessary, antibiotics such as kanamycin, penicillin and streptomycin may be added to the medium during the culturing.

For the culturing of the transformant obtained by using an insect cell as the host cell, generally employed media such as TNM-FH medium (PharMingen, Inc.), Sf-900 II SFM medium (Life Technologies, Inc.), ExCell 400 and ExCell 405 (JRH Biosciences, Inc.) and Grace's Insect Medium [Nature, 195, 788 (1962)] can be used as the medium.

Culturing is usually carried out at pH 6 to 7 at 25 to 30° C. for 1 to 5 days.

If necessary, antibiotics such as gentamicin may be added to the medium during the culturing.

The transformant obtained by using a plant cell as the host cell may be cultured in the form of cells as such or after differentiation into plant cells or plant organs. For the culturing of such transformant, generally employed media such as Murashige-Skoog (MS) medium and White medium, media prepared by adding phytohormones such as auxin and cytokinin to these media, etc. can be used as the medium.

Culturing is usually carried out at pH 5 to 9 at 20 to 40° C. for 3 to 60 days.

If necessary, antibiotics such as kanamycin and hygromycin may be added to the medium during the culturing.

γ-Glutamylcysteine synthetase may be produced by intracellular production, extracellular secretion or production on outer membranes by cells. These methods can be applied by changing the cells used and altering the structure of the protein to be produced.

When γ-glutamylcysteine synthetase is produced in cells or on outer membranes of cells, it is possible to force the protein to be secreted outside the cells by applying the method of Paulson, et al. [J. Biol. Chem., 264, 17619 (1989)], the method of Lowe, et al. [Proc. Natl. Acad. Sci. USA, 86, 8227 (1989); Genes Develop., 4, 1288 (1990)], or the methods described in Japanese Published Unexamined Patent Application No. 336963/93, WO94/23021, etc.

That is, extracellular secretion of γ-glutamylcysteine synthetase by cells can be caused by producing it in such form that a signal peptide is added upstream of an amino acid sequence containing the active site of γ-glutamylcysteine synthetase by the use of recombinant DNA techniques.

It is also possible to increase the enzyme production by utilizing a gene amplification system using a dihydrofolate reductase gene or the like according to the method described in Japanese Published Unexamined Patent Application No. 227075/90.

Further, γ-glutamylcysteine synthetase can be produced using an animal having an introduced gene (non-human transgenic animal) or a plant having an introduced gene (transgenic plant) constructed by redifferentiation of animal or plant cells carrying the introduced gene.

When the transformant having γ-glutamylcysteine synthetase is an animal or plant, the protein can be produced by raising or culturing the animal or plant in a usual manner, allowing the protein to form and accumulate therein, and recovering the protein from the animal or plant.

Production of γ-glutamylcysteine synthetase using an animal can be carried out, for example, by producing the protein in an animal constructed by introducing the gene according to known methods [Am. J. Clin. Nutr., 63, 639S (1996); Am. J. Clin. Nutr., 63, 627S (1996); Bio/Technology, 9, 830 (1991)].

In the case of an animal, the protein can be produced, for example, by raising a non-human transgenic animal carrying the introduced polynucleotide encoding γ-glutamylcysteine synthetase, allowing γ-glutamylcysteine synthetase to form and accumulate in the animal, and recovering the protein from the animal. The places where γ-glutamylcysteine synthetase is formed and accumulated include milk (Japanese Published Unexamined Patent Application No. 309192/88), egg, etc. of the animal. As the promoter in this process, any promoters capable of functioning in an animal can be used. Preferred promoters include mammary gland cell-specific promoters such as a casein promoter, β casein promoter, β lactoglobulin promoter and whey acidic protein promoter.

Production of γ-glutamylcysteine synthetase using a plant can be carried out, for example, by culturing a transgenic plant darrying the introduced polynucleotide encoding γ-glutamylcysteine synthetase according to known methods [Soshiki Baiyo (Tissue Culture), 20 (1994); Soshiki Baiyo, 21 (1995); Trends Biotechnol., 15, 45 (1997)], allowing the protein to form and accumulate in the plant, and recovering the protein from the plant.

γ-Glutamylcysteine synthetase produced by using the cell or the transformant producing γ-glutamylcysteine synthetase can be isolated and purified by conventional methods for isolating and purifying enzymes.

For example, when γ-glutamylcysteine synthetase is produced in a soluble form in cells, the cells are recovered by centrifugation after the completion of culturing and suspended in an aqueous buffer, followed by disruption using a sonicator, French press, Manton Gaulin homogenizer, Dynomill or the like to obtain a cell-free extract.

A purified protein preparation can be obtained by centrifuging the cell-free extract to obtain the supernatant and then subjecting the supernatant to ordinary means for isolating and purifying enzymes, e.g., extraction with a solvent, salting-out with ammonium sulfate, etc., desalting, precipitation with an organic solvent, anion exchange chromatography using resins such as diethylaminoethyl (DEAE)-Sepharose and DIAION HPA-75 (Mitsubishi Chemical Corporation), cation exchange chromatography using resins such as S-Sepharose FF (Pharmacia), hydrophobic chromatography using resins such as butyl Sepharose and phenyl Sepharose, gel filtration using a molecular sieve, affinity chromatography, chromatofocusing, and electrophoresis such as isoelectric focusing, alone or in combination.

When the protein is produced as an inclusion body in cells, the cells are similarly recovered and disrupted, followed by centrifugation to obtain a precipitate fraction. After the protein is recovered from the precipitate fraction by an ordinary method, the inclusion body of the protein is solubilized with a protein-denaturing agent.

The solubilized protein solution is diluted with or dialyzed against a solution containing no protein-denaturing agent or a solution containing the protein-denaturing agent at such a low concentration that denaturation of protein is not caused, whereby the protein is renatured to have normal higher-order structure. Then, a purified protein preparation can be obtained by the same isolation and purification steps as described above.

When γ-glutamylcysteine synthetase or its derivative such as a glycosylated form is extracellularly secreted, the protein or its derivative such as a glycosylated form can be recovered in the culture supernatant.

That is, the culture is treated by the same means as above, e.g., centrifugation, to obtain a soluble fraction. A purified protein preparation can be obtained from the soluble fraction by using the same isolation and purification methods as described above.

An example of γ-glutamylcysteine synthetase obtained in the above manner is a protein having the amino acid sequence of SEQ ID NO: 1.

It is also possible to produce γ-glutamylcysteine synthetase as a fusion protein with another protein and to purify it by affinity chromatography using a substance having affinity for the fused protein. For example, the polypeptide of the present invention can be produced as a fusion protein with protein A and can be purified by affinity chromatography using immunoglobulin G according to the method of Lowe, et al. [Proc. Natl. Acad. Sci. USA, 86, 8227 (1989); Genes Develop., 4, 1288 (1990)] and the methods described in Japanese Published Unexamined Patent Application No. 336963/93 and WO94/23021.

γ-Glutamylcysteine synthetase can also be produced as a fusion protein with a Flag peptide and purified by affinity chromatography using an anti-Flag antibody [Proc. Natl. Acad. Sci. USA, 86, 8227 (1989); Genes Develop., 4, 1288 (1990)]. Further, the protein can be purified by affinity chromatography using an antibody against γ-glutamylcysteine synthetase.

γ-Glutamylcysteine synthetase can also be produced by chemical synthetic methods such as the Fmoc method (the fluorenylmethyloxycarbonyl method) and the tBoc method (the t-butyloxycarbonyl method) based on the amino acid information on γ-glutamylcysteine synthetase. Further, γ-glutamylcysteine synthetase can be chemically synthesized by using peptide synthesizers from Advanced ChemTech, Perkin-Elmer, Pharmacia, Protein Technology Instrument, Synthecell-Vega, PerSeptive, Shimadzu Corporation, etc.

### 6. Process for Preparing a Culture of the Cells Having γ-Glutamylcysteine Synthetase and Treated Matters of the Culture Used in the Present Invention

A culture of the cells having γ-glutamylcysteine synthetase used in the present invention can be obtained by culturing the cells of the above 3 or the transformant obtained in the above 5(1) and (2) by the method described in the above 5(3) and allowing γ-glutamylcysteine synthetase to form and accumulate in the culture.

Examples of the treated culture of the cells having γ-glutamylcysteine synthetase used in the present invention include products obtained by subjecting the culture to concentration and drying, cells obtained by centrifuging the culture, products obtained by subjecting the cells to drying, freeze-drying, treatment with a surfactant, treatment with a solvent and enzymatic treatment, living cells such as a product obtained by subjecting the cells to immobilization, products obtained by subjecting the cells to ultrasonication, mechanical friction and protein fractionation, and crude enzyme extracts obtained from the cells, such as an enzyme preparation. They can be prepared by known methods so far as they can be used as an enzyme source in the production process of the present invention, that is, they have γ-glutamylcysteine synthetase activity.

### 7. Process for Producing the γ-Glutamylamide Compounds of the Present Invention

### (1) Enzymatic Production Process

The γ-glutamylamide compound represented by formula (I) (wherein R¹ and R², which may be the same or different, each represent a hydrogen atom, substituted or unsubstituted lower alkyl, substituted or unsubstituted lower alkenyl, or substituted or unsubstituted lower alkynyl, but are not a hydrogen atom at the same time) can be produced by forming the γ-glutamylamide compound from a glutamyl donor and an amine compound using γ-glutamylcysteine synthetase as an enzyme source.

More specifically, the γ-glutamylamide compound is produced by allowing γ-glutamylcysteine synthetase, a glutamyl donor, an amine compound and ATP to be present in an aqueous medium, allowing the γ-glutamylamide compound to form and accumulate in the medium and recovering the γ-glutamylamide compound from the medium.

In the above production process of the present invention, there is no particular restriction as to the glutamyl donor used as a substrate so long as it serves as a substrate for γ-glutamylcysteine synthetase and reacts with an amine compound to give the γ-glutamylamide compound, and, for example, L-glutamic acid, D-glutamic acid and 2-oxoglutaric acid and their salts, preferably L-glutamic acid and its salts can be used.

The amine compounds include amine compounds represented by the following formula (III):

HNR¹R² (III)

(wherein R¹ and R² have the same meanings as defined above).

Examples of the amine compounds represented by formula (III) are preferably methylamine, ethylamine, propylamine, cyclopropylamine and butylamine, more preferably ethylamine.

In the above process, γ-glutamylcysteine synthetase is usually added in an amount of 0.01 to 100 mg, preferably 0.1 mg to 10 mg per g of glutamyl donor used as a substrate.

In the above process, the glutamyl donor and the amine compound used as substrates are added to the aqueous medium at the start or in the course of reaction to give a concentration usually of 0.1 to 500 g/l, preferably 0.2 to 200 g/l.

In the above process, ATP used as an energy source is usually used at a concentration of 0.5 mmol/l to 10 mol/l.

The aqueous medium used in the process of the present invention may comprise any components and may have any composition so far as the γ-glutamylamide compound-forming reaction is not inhibited. Suitable aqueous media include water, buffers such as phosphate buffer, carbonate buffer, acetate buffer, borate buffer, citrate buffer and Tris buffer, alcohols such as methanol and ethanol, esters such as ethyl acetate, ketones such as acetone, and amides such as acetamide.

The γ-glutamylamide compound-forming reaction is carried out in the aqueous medium usually at pH 5 to 11, preferably pH 6 to 10, at 20 to 50° C., preferably 25 to 45° C., for 2 to 150 hours, preferably 6 to 120 hours.

The γ-glutamylamide compounds produced by the above process include compounds represented by formula (I) (wherein R¹ and R² have the same meanings as defined above), preferably those in which R¹ of formula (I) is a hydrogen atom and R² is methyl, ethyl, propyl, cyclopropyl or butyl, more preferably theanine represented by formula (II).

### (2) Production Process Using a Culture of the Cells, Etc. as an Enzyme Source

The γ-glutamylamide compound of formula (I) (wherein R¹ and R² have the same meanings as defined above) can be produced by forming the γ-glutamylamide compound from a glutamyl donor and an amine compound using a culture of the cells having γ-glutamylcysteine synthetase or a treated culture as an enzyme source.

More specifically, the γ-glutamylamide compound can be produced by [1] a process which comprises allowing a culture of the cells having γ-glutamylcysteine synthetase or a treated culture and an amine compound to be present in an aqueous medium, allowing a γ-glutamylamide compound to form and accumulate in the medium and recovering the γ-glutamylamide compound from the medium; and [2] a process which comprises allowing a culture of the cells having γ-glutamylcysteine synthetase or a treated culture, a glutamyl donor and an amine compound to be present in an aqueous medium, allowing a γ-glutamylamide compound to form and accumulate in the medium and recovering the γ-glutamylamide compound from the medium.

The culture of the cells having γ-glutamylcysteine synthetase or the treated culture used in the above process includes cultures and the like that can be prepared according to the method of the above 6.

In the above process, the kinds of the substrates and the concentration thereof to be used, as well as the γ-glutamylamide compounds produced, are the same as those in the enzymatic production process of the above 7(1).

As the aqueous medium used in the above process, a culture liquor of the cells used as the enzyme source can be used in addition to the aqueous media used in the enzymatic production process of the above 7(1).

Further, in the above process, compounds which can be metabolized by the cells to produce ATP, for example, sugars such as glucose, alcohols such as ethanol, and organic acids such as acetic acid may be added, as ATP source, to the aqueous medium according to need.

If necessary, a surfactant or an organic solvent may further be added to the aqueous medium. Any surfactant that promotes the formation of a galactose-containing complex carbohydrate can be used. Suitable surfactants include nonionic surfactants such as polyoxyethylene octadecylamine (e.g., Nymeen S-215, NOF Corporation), cationic surfactants such as cetyltrimethylammonium bromide and alkyldimethylbenzylammonium chloride (e.g., Cation F2-40E, NOF Corporation), anionic surfactants such as lauroyl sarcosinate, and tertiary amines such as alkyldimethylamine (e.g., Tertiary Amine FB, NOF Corporation), which may be used alone or in combination. The surfactant is usually used at a concentration of 0.1 to 50 g/l. As the organic solvent, xylene, toluene, aliphatic alcohols, acetone, ethyl acetate, etc. may be used usually at a concentration of 0.1 to 50 ml/l.

When a culture or a treated culture is used as the enzyme source, the amount of the enzyme source to be added varies according to its specific activity, etc., but is, for example, 5 to 1000 mg, preferably 10 to 400 mg per mg of glutamyl donor used as a substrate.

The γ-glutamylamide compound-forming reaction is carried out in the aqueous medium usually at pH 5 to 11, preferably pH 6 to 10, usually at 20 to 50° C., preferably 25 to 45° C., usually for 2 to 150 hours, preferably 6 to 120 hours.

Recovery of the γ-glutamylamide compound formed and accumulated in the aqueous medium can be carried out by ordinary methods using active carbon, ion-exchange resins, etc. or by means such as extraction with an organic solvent, crystallization, thin layer chromatography and high performance liquid chromatography.

Certain embodiments of the present invention are illustrated in the following examples. These examples are not to be construed as limiting the scope of the invention.

### EXAMPLE 1

### Construction of a Strain Expressing γ-Glutamylcysteine Synthetase

Escherichia coli has the ability to form glutathione and the gene encoding γ-glutamylcysteine synthetase, as an enzyme involved in the biosynthesis of glutathione, has been identified [Nucleic Acids Res., 14, 4393-400 (1986)].

Accordingly, a strain expressing γ-glutamylcysteine synthetase was constructed by cloning a polynucleotide encoding γ-glutamylcysteine synthetase by the following method.

First, the chromosomal DNA of Escherichia coli W3110 was isolated and purified by the method using saturated phenol described in Current Protocols in Molecular Biology.

By using a DNA synthesizer (Model 8905, PerSeptive Biosystems, Inc.), DNAs having the nucleotide sequences of SEQ ID NOS: 3 to 6 (hereinafter referred to as primer A, primer B, primer C and primer D, respectively) were synthesized. Primer A has a nucleotide sequence wherein a nucleotide sequence containing the HindIII recognition sequence is added to the 5′ end of a region containing the initiation codon of the known γ-glutamylcysteine synthetase gene of Escherichia coli. The initiation codon of this gene, which begins with TTG, was altered to begin with ATG in expectation of improved translation efficiency. Primer B has a nucleotide sequence wherein a nucleotide sequence containing the BamHI recognition sequence is added to the 5′ end of a nucleotide sequence complementary to a sequence containing the termination codon of the γ-glutamylcysteine synthetase gene. Primer C has a nucleotide sequence wherein a nucleotide sequence containing the EcoRI recognition sequence is added to the 5′ end of the nucleotide sequence of the lac promoter region of expression vector pUC19. Primer D has a nucleotide sequence wherein a nucleotide sequence containing the HindIII recognition sequence is added to the 5′ end of a sequence complementary to the sequence of the lac promoter region of expression vector pUC19.

PCR was carried out using the above primer A and primer B and, as a template, the chromosomal DNA of Escherichia coli W3110 for amplification of a polynucleotide fragment encoding γ-glutamylcysteine synthetase, and primer C and primer D and, as a template, pUC19 for amplification of a lac promoter region fragment.

PCR was carried out for 30 cycles of 94° C. for one minute, 55° C. for 2 minutes and 72° C. for 3 minutes, using 40 μl of a reaction mixture comprising 0.1 μg of the chromosomal DNA or 100 ng of pUC19 as a template, 0.5 μmol/l each of the primers, 2.5 units of Pfu DNA polymerase (Stratagene), 4 μl of buffer for Pfu DNA polymerase (10×) (Stratagene) and 200 μmol/l each of dNTPs (dATP, dGTP, dCTP and TTP).

One-tenth of each of the resulting reaction mixtures was subjected to agarose gel electrophoresis to confirm that a ca. 1.6 kb polynucleotide fragment corresponding to the polynucleotide fragment encoding γ-glutamylcysteine synthetase was amplified by PCR using primer A and primer B, and a ca. 0.3 kb polynucleotide fragment corresponding to the lac promoter region was amplified by PCR using primer C and primer D. Then, the remaining reaction mixture was mixed with an equal amount of phenol/chloroform (1 vol/l vol) saturated with TE [10 mmol/l Tris-HCl (pH 8.0), 1 mmol/l EDTA]. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained DNA solutions (5 μl each) were respectively subjected to reaction to cleave the polynucleotide fragment encoding γ-glutamylcysteine synthetase with restriction enzymes HindIII and BamHI and to reaction to cleave the lac promoter region DNA with restriction enzymes HindIII and EcoRI. DNA fragments were separated by agarose gel electrophoresis, and a 1.6 kb DNA fragment containing the polynucleotide encoding γ-glutamylcysteine synthetase and a 0.3 kb DNA fragment containing the lac promoter region were respectively recovered using GENECLEAN II Kit (BIO 101).

Expression vector pTrS33 (Japanese Patent No. 2928287) (0.2 μg) was cleaved with restriction enzymes HindIII and EcoRI. After treatment with alkaline phosphatase, DNA fragments were separated by agarose gel electrophoresis, and a 3.16 kb DNA fragment was recovered in the same manner as above.

The 0.3 kb fragment containing the lac promoter region and the 3.16 kb vector fragment obtained above were subjected to ligation reaction using a ligation kit (Takara Shuzo Co., Ltd.) at 16° C. for 16 hours.

Escherichia coli DH5a (Toyobo Co., Ltd.) was transformed using the ligation reaction mixture by the method using calcium ion [Proc. Natl. Acad. Sci. USA, 69, 2110 (1972), and the resulting transformant was spread on LB agar medium containing 50 μg/ml ampicillin and cultured overnight at 30° C.

A plasmid was extracted from a colony of the transformant that grew on the medium according to a known method, and it was confirmed that an expression vector into which the lac promoter was inserted was obtained. The expression vector was designated as pTrS33L.

pTrS33L was cleaved with restriction enzymes HindIII and BamHI. After treatment with alkaline phosphatase, a polynucleotide fragment was separated by agarose gel electrophoresis, and a 2.5 kb polynucleotide fragment was recovered in the same manner as above.

The 1.6 kb fragment containing and the 2.5 kb vector fragment obtained above were subjected to ligation reaction using a ligation kit (TAKARA BIO INC.) at 16° C. for 16 hours.

Escherichia coli DH5a (Toyobo Co., Ltd.) was transformed using the ligation reaction mixture according to the method using calcium ion [Proc. Natl. Acad. Sci. USA, 69, 2110 (1972), and the resulting transformant was spread on LB agar medium containing 50 μg/ml ampicillin and cultured overnight at 30° C.

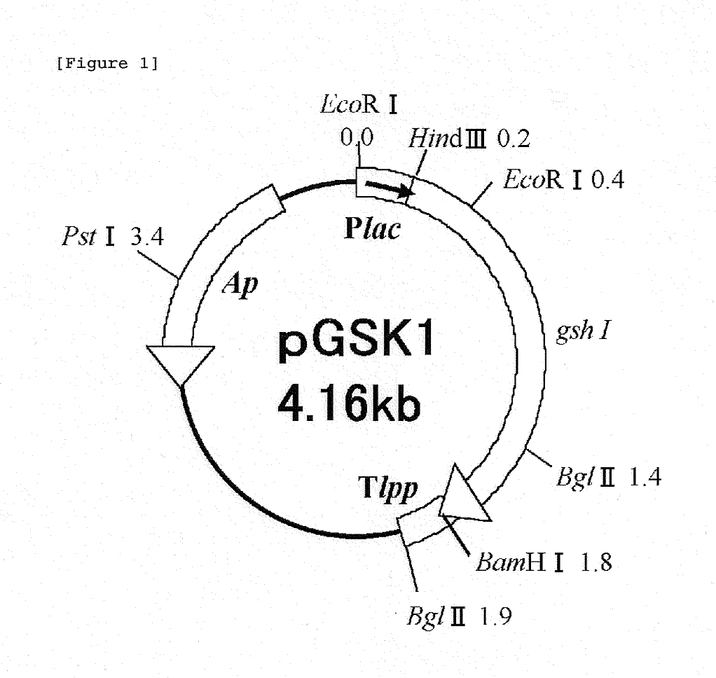

A plasmid was extracted from a colony of the transformant that grew on the medium according to a known method. By sequence determination and restriction enzyme digestion, it was confirmed that a plasmid DNA in which the polynucleotide encoding γ-glutamylcysteine synthetase, which encodes a protein having the amino acid sequence of SEQ ID NO: 1 and which has the nucleotide sequence of SEQ ID NO: 2, was ligated downstream of the lac promoter was obtained, and the plasmid DNA was designated as pGSK1. The structure of pGSK1 is shown in FIG. 1. Also, Escherichia coli DH5a carrying the plasmid was designated as Escherichia coli DH5a/pGSK1.

### EXAMPLE 2

### Production of a γ-Glutamylamide Compound Using a Treated Matter of Culture as the Enzyme Source (1)

Recombinant Escherichia coli DH5a/pGSK1 constructed in Example 1 was inoculated into 40 ml of LB medium [10 g/l Bacto-tryptone (Difco), 5 g/l yeast extract (Difco) and 5 g/l NaCl] containing 100 mg/l ampicillin, and subjected to shaking culture in a 300-ml Erlenmeyer flask at 30° C. overnight. After the completion of culturing, the cells recovered by centrifugation of the culture were suspended in 100 mmol/l Tris-HCl (pH 8.0) and the cell concentration was adjusted so that OD660 became 70 as measured by a spectrophotometer, whereby a cell-containing solution was obtained.

To the cell-containing solution was added xylene at a concentration of 10 ml/l, and the mixture was vortexed for 15 minutes to obtain treated cells. To 100 μl of the treated cells were added various substances at the concentrations shown in Table 1, and the resulting reaction mixture (total volume: 200 μl) was reacted at 37° C. for 60 minutes.

TABLE 1
Composition of Reaction Mixture

| | | |
|---|---|---|
| Tris | 12.1 | g/l |
| MgSO₄•7H₂O | 5 | g/l |
| K₂SO₄ | 3.5 | g/l |
| NAD•3H₂O | 143 | mg/l |
| FMN•Na•2H₂O | 65.6 | mg/l |
| ATP | 12.1 | g/l |
| Sodium glutamate | 0 or 2.5 | g/l |
| Ethylamine hydrochloride | 0 or 20 | g/l |

After the completion of reaction, theanine formed in the reaction mixture was detected and quantitatively determined by HPLC analysis of the reaction mixture under the following conditions. The results are shown in Table 2.

Conditions for HPLC analysis:

Mobile phase: 3.5% aqueous solution containing 2 g/l acetonitrile and sodium 1-heptanesulfonate (adjusted to pH 2.0 with phosphoric acid)

Column: two Nucreosil columns (GL Sciences, 4.6×150 mm) connected; column temperature: 40° C.

Flow rate: 0.9 ml/minute

Detection: absorption at 210 nm

TABLE 2

| Amount of | Amount of L- | Amount of theanine |
|---|---|---|
| ethylamine added | glutamic acid | formed |
| (g/l) | added (g/l) | (mg/l) |
| 0 | 0 | 0 |
| 20 | 2.5 | 520 |
| 20 | 0 | 54 |

As shown above, it was found that theanine can be produced by using, as the enzyme source, a treated culture of Escherichia coli DH5a/pGSK1 obtained by introducing the polynucleotide encoding γ-glutamylcysteine synthetase. Further, as formation of theanine was confirmed without the addition of L-glutamic acid as a substrate, it was also found that cells in which the ability to produce L-glutamic acid, which serves as a glutamyl donor, is not particularly enhanced have the ability to produce a γ-glutamylamide compound such as theanine only by adding an amine compound as a substrate.

### EXAMPLE 3

### Production of a γ-Glutamylamide Compound Using a Treated Matter of Culture as the Enzyme Source (2)

Recombinant plasmid pGSK1 into which the polynucleotide encoding γ-glutamylcysteine synthetase was inserted was introduced into Escherichia coli BL21 (Takara Shuzo Co., Ltd.) according to an ordinary method to obtain Escherichia coli BL21/pGSK1.

Escherichia coli BL21/pGSK1 was spread on LB agar medium containing 100 mg/l ampicillin and subjected to static culture at 30° C. overnight. The cells that grew on the medium were inoculated into 300 ml of a pre-culture medium [6% corn steep liquor, 1.15% sodium glutamate, 0.2% lactic acid, 200 mg/l Casamino acid, 5 mg/l vitamin B1 (pH 7.2)] in a 2-1 Erlenmeyer flask and cultured at 28° C. at 220 rpm for 20 hours. The obtained culture (2.25 ml) was inoculated into one liter of a seed medium [2% corn steep liquor, 0.5% soybean peptide (SMS: Fuji Oil Co., Ltd.), 1.5% dipotassium hydrogenphosphate, 0.1% sodium chloride, 0.6% ammonium sulfate, 0.1% glycine, 0.06% arginine hydrochloride, 4.95 mg/l ferrous sulfate, 4.4 mg/l zinc sulfate, 1.97 mg/l copper sulfate, 360 μg/1 manganese chloride, 440 μg/l sodium borate, 185 μg/l ammonium molybdate, 5 mg/l vitamin B1, 5 mg/l nicotinic acid, 20 mg/l leucine, 20 mg/l threonine, 20 mg/l tryptophan, 0.01% LG109 (Asahi Denka), 1% glucose, 0.05% magnesium sulfate, 100 mg/l ampicillin (pH 6.5)] in a 2-l jar and cultured with aeration at a rate of 1 l/minute and agitation at a speed of 800 rpm at 30° C. for 8 hours.

The obtained culture (28 ml) was inoculated into one liter of a production medium [2.25% corn steep liquor, 0.55% soybean peptide (SMS: Fuji Oil Co., Ltd.), 1.68% dipotassium hydrogenphosphate, 0.115% sodium chloride, 0.68% ammonium sulfate, 5.57 mg/l ferrous sulfate, 4.95 mg/l zinc sulfate, 2.21 mg/l copper sulfate, 405 μg/1 manganese chloride, 495 μg/l sodium borate, 208 μg/l ammonium molybdate, 5.6 mg/l vitamin B1, 5.6 mg/l nicotinic acid, 22 mg/l leucine, 22 mg/l threonine, 22 mg/l tryptophan, 0.018% LG109, 1.26% glucose, 0.08% magnesium sulfate, 100 mg/l ampicillin (pH 6.5)] in a 2-l jar and cultured with aeration at a rate of 1 l/minute and agitation at a speed of 800 rpm at 30° C. while controlling the pH to 6.5 with 28% aqueous ammonia. During the culturing, 340 ml of a sugar solution (57.7% glucose, 0.188 g/l calcium chloride) for feeding was added at a fixed flow rate.

Culturing was terminated after 30 hours and 7.5 ml/l xylene was added, followed by agitation for 10 minutes to obtain a treated culture. To 700 ml of the treated culture were added 3.5 g of magnesium sulfate, 2.45 g of potassium sulfate, 350 mg of ATP, 50 mg of NAD, 22 mg of FMN, 65 g of glucose, 28 g of ethylamine hydrochloride and 56 g of sodium glutamate, and the mixture was reacted with aeration at a rate of 0.7 ml/minute and agitation at a speed of 950 rpm at 34° C. for 18 hours while controlling the pH to 7.2 with sodium hydroxide solution. The reaction product was analyzed under the same conditions as those of Example 2 to confirm that 2.1 g/l theanine was formed.

### INDUSTRIAL APPLICABILITY

In accordance with the present invention, γ-glutamylamide compounds, preferably theanine can be produced simply and efficiently.

### SEQUENCE LISTING FREE TEXT

### SEQ ID NO: 3—Description of Artificial Sequence: Synthetic DNA

### SEQ ID NO: 4—Description of Artificial Sequence: Synthetic DNA

### SEQ ID NO: 5—Description of Artificial Sequence: Synthetic DNA

SEQ ID NO: 6—Description of Artificial Sequence: Synthetic DNA

1. A process for producing a γ-glutamylamide compound represented by formula (I): 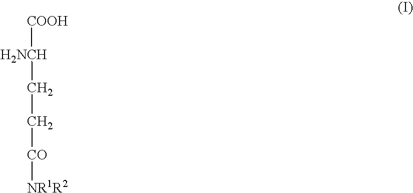
(wherein R¹ and R², which may be the same or different, each represent a hydrogen atom, substituted or unsubstituted lower alkyl, substituted or unsubstituted lower alkenyl, or substituted or unsubstituted lower alkynyl, but are not hydrogen atoms at the same time), which comprises forming the γ-glutamylamide compound from a glutamyl donor and an amine compound using γ-glutamylcysteine synthetase, or a culture of cells having the enzyme or a treated culture of the cells as an enzyme source.

2. The process according to claim 1, wherein the γ-glutamylcysteine synthetase is an enzyme derived from a microorganism.

3. The process according to claim 2, wherein the microorganism is any of an enteric bacterium, yeast and a filamentous fungus.

4. The process according to claim 2, wherein the microorganism is a microorganism belonging to the genus Escherichia.

5. The process according to claim 1, wherein the γ-glutamylcysteine synthetase is a protein according to any of the following [1] to [3]:
[1] a protein having the amino acid sequence of SEQ ID NO: 1;
[2] a protein consisting of an amino acid sequence wherein one or more amino acid residues are deleted, substituted or added in the amino acid sequence of SEQ ID NO: 1 and having γ-glutamylcysteine synthetase activity; and
[3] a protein consisting of an amino acid sequence which has 80% or more homology to the amino acid of SEQ ID NO: 1.

6. The process according to claim 1, wherein the cell is a microorganism.

7. The process according to claim 6, wherein the microorganism is any of an enteric bacterium, yeast and a filamentous fungus.

8. The process according to claim 6, wherein the microorganism is a microorganism belonging to the genus Escherichia.

9. The process according to any one of claims 1 and 6 to 8, wherein the cell having γ-glutamylcysteine synthetase is a cell into which a polynucleotide encoding γ-glutamylcysteine synthetase has been introduced.

10. The process according to claim 9, wherein the polynucleotide encoding γ-glutamylcysteine synthetase is a polynucleotide according to any of the following:
[1] a polynucleotide encoding a protein having the amino acid sequence of SEQ ID NO: 1; and
[2] a polynucleotide having the nucleotide sequence of SEQ ID NO: 2

11. The process according to claim 9, wherein the polynucleotide encoding γ-glutamylcysteine synthetase is a polynucleotide encoding a protein consisting of an amino acid sequence wherein one or more amino acid residues are deleted, substituted or added in the amino acid sequence of SEQ ID NO: 1.

12. The process according to claim 9, wherein the polynucleotide encoding γ-glutamylcysteine synthetase is a polynucleotide encoding a protein consisting of an amino acid sequence which has 80% or more homology to the amino acid of SEQ ID NO: 1.

13. The process according to claim 9, wherein the polynucleotide encoding γ-glutamylcysteine synthetase is a polynucleotide which hybridizes with a polynucleotide having a nucleotide sequence complementary to the nucleotide sequence of SEQ ID NO: 2 under conditions of washing in 0.2×SSC solution at 65° C.

14. The process according to claim 9, wherein the polynucleotide encoding γ-glutamylcysteine synthetase is a polynucleotide comprising a sequence having at least 90% homology to SEQ ID NO:2.


SEQUENCE LISTING (6 sequences)

SEQ ID NO: 1 - protein, 518 aa, Escherichia coli W3110
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 2 - DNA, 1554 bp, Escherichia coli W3110
ttgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcc

SEQ ID NO: 3 - DNA, 33 bp, Artificial Sequence
  Description of Artificial Sequence Synthetic DNA
cgggagaagcttatgatcccggacgtatcacag

SEQ ID NO: 4 - DNA, 32 bp, Artificial Sequence
  Description of Artificial Sequence Synthetic DNA
ttttctggatccttaggcgtgtttttccagcc

SEQ ID NO: 5 - DNA, 30 bp, Artificial Sequence
  Description of Artificial Sequence Synthetic DNA
ggaagagaattcaatacgcaaaccgcctct

SEQ ID NO: 6 - DNA, 32 bp, Artificial Sequence
  Description of Artificial Sequence Synthetic DNA
ggtcataagcttttcctgtgtgaaattgttat